(19) United States
(12) Patent Application Publication
(10) Pub. No.: US 2018/0357929 A1
(43) Pub. Date: Dec. 13, 2018

(54) System and Method for Part-Task Training Box for Flexible Endoscopy

(71) Applicant: Brigham and Women's Hospital, Inc., Boston, MA (US)

(72) Inventors: Christopher C. Thompson, Needham, MA (US); Amy Ou, Cambridge, MA (US); Balazs Lengyel, Tucson, AZ (US); Andrew Camacho, Miami, FL (US); Pichamol Jirapinyo, Hamden, CT (US)

(21) Appl. No.: 16/057,003

(22) Filed: Aug. 7, 2018

Related U.S. Application Data
(63) Continuation of application No. 14/364,234, filed on Jun. 10, 2014.
(60) Provisional application No. 61/570,430, filed on Dec. 14, 2011.

(51) Int. Cl. G09B 23/28; A61B 1/00

(52) U.S. Cl. CPC G09B 23/285; A61B 1/00131

(57) ABSTRACT

Embodiments of the invention provide an endoscopic training system and method for the development of endoscopic procedure skills and technical competency of an endoscopist. The system includes a portable part-task enclosure with a plurality of walls defining an internal chamber. One of the walls includes an access port coupling the internal chamber to an external environment and the access port can be configured to facilitate the passage of a flexible endoscope insertion member. The system further includes a plurality of objects disposed within the internal chamber. Each of the plurality of objects is configured for manipulation by a medical tool that extends through the flexible endoscope insertion member.

### CROSS REFERENCE TO RELATED APPLICATIONS

This application is a continuation of U.S. patent application Ser. No. 14/364,234 filed Jun. 10, 2014, which represents the national stage entry of International Application PCT/US2012/069763 filed Dec. 14, 2012, which claims benefit of U.S. Provisional Application 61/570,430 filed Dec. 14, 2011, all of which are hereby incorporated by reference in their entirety for all purposes.

### BACKGROUND OF THE INVENTION

Endoscopy is a minimally invasive diagnostic medical procedure used to view interior parts of the body, such as the interior or exterior surfaces of organs, joints or cavities. A flexible endoscope is used to view interior parts of the body and has a moveable tip with an objective lens, a flexible shaft and a handle. Light is usually transferred from an external light source via glass fiber bundles to the moveable tip, where the light is emitted to illuminate the bodily features to be viewed. The illuminated bodily feature is projected by an objective lens onto a separate thin bundle of glass fibers, each fiber diameter typically ranging from 4 μm to 18 μm and the number of fibers from approximately 3,000 to over 20,000. This fiberoptic bundle optically transfers the image to the handle of the flexible endoscope where it is enlarged and made visible by an eyepiece lens. The fiberoptic bundles are extremely flexible, and an image can be transmitted even when tied in a knot.

The movable tip also includes an operating channel which allows the passage of additional instruments, such as forceps or snares, to be inserted into the endoscope handle and passed along the length of the endoscope shaft. The instrument emerges from the distal end of the endoscope into the field of view. An endoscopist can then use various endoscopic maneuvers to navigate the different instruments to achieve therapeutic and diagnostic results.

One example of an additional instrument which can be inserted through the operating channel of the flexible endoscope is a rat tooth forcep. Rat tooth forceps have interdigitating teeth and are typically used to hold skin or dense tissue without slipping or to retrieve stones, stents or other objects from inside body cavities. Another example of an additional instrument is a polypectomy snare which is typically used during colonoscopy procedures to remove polyps. The loop of the polypectomy snare is tightened and pulled away to grip the stalk of the polyp attached to the intestinal wall. An electric current is then passed through the snare loop to cut through the polyp, while simultaneously providing electrocautery.

Unfortunately, the skills used to perform these specific endoscopic maneuvers with different instruments takes years of practice. This is the case because typical methods for steering the endoscope tip are unusual. That is, the movable tip is controlled by pull wires attached at the moveable tip just beneath the outer protective shaft, and passing back through the length of the shaft to angling control knobs in the handle. One control knob, typically a larger control knob, moves the tip in an upward or downward direction, whereas a second control knob, typically a smaller knob, moves the tip from left to right. Simultaneously adjusting the control knobs and maneuvering the endoscope can be extremely challenging.

Currently there is no simple and affordable training device or simulator for developing the above skills and assessing endoscopic skills. Many of the current simulators use virtual environments where CT or MR images are processed by a computer to reconstruct a three-dimensional environment similar to that seen through an endoscope. Based on physical movement of an endoscope by the trainee, the computer program changes the view on the screen to simulate the endoscopic procedure. These systems have many shortcomings. They simulate entire procedures without the ability to identify a specific technical weakness or to focus on one aspect of a procedure with which a trainee may have difficulty. Additionally, these virtual training systems are typically rather expensive and most centers do not have the resources to purchase or maintain the systems. Thus, a trainee's initial clinical procedure is often the first time they will have handled an endoscope. Therefore, an affordable system is needed for the development of endoscopic skills and the objective assessment of basic technical competency.

### SUMMARY OF THE INVENTION

The present invention relates to an affordable endoscopic training system for the development of endoscopic procedure skills and the assessment of technical competency of an endoscopist. It also allows for training, development, and evaluation of essential endoscopic part-tasks that are critical in the clinical performance of colonoscopy and upper endoscopy.

Some embodiments of the invention provide a system which includes a portable part-task enclosure with a plurality of walls defining an internal chamber. One of the walls includes an access port coupling the internal chamber to an external environment and the access port is configured to facilitate the passage of a flexible endoscope insertion member. The system further includes a plurality of objects disposed within the internal chamber. Each of the plurality of objects is configured for manipulation by a medical tool that extends through the flexible endoscope insertion member.

In another embodiment, the invention provides a method for the development of endoscopic procedure skills and the assessment of technical competency of an endoscopist. The method includes providing a portable part-task enclosure having a plurality of walls defining an internal chamber. The internal chamber has a plurality of objects disposed therein, and the portable part-task enclosure has an access port which couples the internal chamber to an external environment. The method also includes inserting a flexible endoscope insertion member through the access port and into the internal chamber and maneuvering the flexible endoscope insertion member within the internal chamber. The method further provides delivering a medical tool to the internal chamber via the flexible endoscope insertion member and manipulating one of the plurality of objects within the internal chamber using the medical tool.

In another embodiment, the invention provides an endoscopic training kit for development of endoscopic procedure skills and assessment of technical competency of an endoscopist. The endoscopic training kit includes a first portable part-task enclosure having a first plurality of walls that define a first internal chamber. At least one of the plurality of walls includes a first access port coupling the internal chamber to an external environment, and the first access port is configured to facilitate passage of a flexible endoscope insertion member. A plurality of objects are disposed within the first internal chamber. The plurality of objects are each configured for manipulation by a medical tool extending through the flexible endoscope insertion member. A second portable part-task enclosure has a second plurality of walls that define a second internal chamber. At least one of the second plurality of walls includes a second access port coupling the second internal chamber to the external environment. The second access port is configured to facilitate passage of the flexible endoscope insertion member. An object is disposed within the second internal chamber, and the object defines a passageway for maneuvering therethrough by the flexible endoscope insertion member.

These and other features, aspects, and advantages of the present invention will become better understood upon consideration of the following detailed description, drawings, and appended claims.

### BRIEF DESCRIPTION OF THE DRAWINGS

FIG. 1 is a perspective view of an exemplary flexible endoscope system that may be used with an endoscope training system according to one embodiment of the present invention.

FIG. 2 is a detailed perspective view of the control head and movable end of the flexible endoscope system of FIG. 1.

FIG. 3 is a detailed perspective view of the movable end of a flexible endoscope insertion member of FIG. 1 including a medical tool channel.

FIG. 4 is a perspective view of a polypectomy snare medical tool that may be deployed via the endoscope insertion member.

FIG. 5 is a perspective view of a rat tooth forcep medical tool that may be deployed via the endoscope insertion member.

FIG. 6 is a top perspective view of an endoscopic training system for development of navigation and loop reduction skills according to one embodiment of the invention.

FIG. 7A is a side perspective view of an endoscopic training system for development of retroflexion skills according to one embodiment of the invention.

FIG. 7B is a side cross-sectional view taken along line 7B-7B of FIG. 7A.

FIG. 7C is a side cross-sectional view taken along line 7C-7C of FIG. 7A.

FIG. 7D is a side cross-sectional view taken along line 7D-7D of FIG. 7A.

FIG. 7E is a top perspective view of the training system of FIG. 7A.

FIG. 7F is an additional top perspective view of FIG. 7A.

FIG. 8A is side perspective view of an endoscopic training system for development of torquing skills according to one embodiment of the invention.

FIG. 8B is a side cross-sectional view taken along line 8B-8B of FIG. 8A.

FIG. 8C is a top perspective view of the training system of FIG. 8A.

FIG. 9A is a perspective view of an endoscopic training system for development of tip deflection skills according to one embodiment of the invention.

FIG. 9B is a top perspective view of the training system of FIG. 9A.

FIG. 9C is a side cross-sectional view taken along line 9C-9C of FIG. 9A.

FIG. 10A is a perspective view of an endoscopic training system for development of polypectomy skills according to one embodiment of the invention.

FIG. 10B is a perspective view of the second enclosure of FIG. 10A.

FIG. 11 is a perspective view of multiple endoscopic training systems included in one enclosure according to one embodiment of the invention.

FIG. 12 is a detailed perspective view of multiple endoscopic training systems included in one enclosure of FIG. 11 with transparent walls.

FIG. 13 is a bottom view of the single enclosure of FIG. 11 with transparent walls.

FIG. 14 is a side cross-sectional view taken along line 14-14 of FIG. 11 to show the compression lock mechanism of the endoscopic training system for development of tip deflection skills.

FIG. 15 is a front perspective view of a portable part-task enclosure according to one embodiment of the invention.

FIG. 16 is a rear perspective view of a portable part-task enclosure according to one embodiment of the invention.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

The present invention relates to multiple tasks, each of which focuses on specific endoscopic maneuvers. The tasks were developed based on important differences in technique and performance exhibited between expert and novice endoscopists. The present invention also relates to a tool to objectively assess and train basic endoscopic skills and technical competency that requires no computerized simulation. The training and assessment methods use all of the five embodiments described below and shown in the figures. However, it may be beneficial to only use one or a subset of the embodiments.

Different medical and surgical specialties (such as Gastroenterology, Urology, Otolaryngology, Gynecology, Cardiology, Vascular Surgery, and Interventional Radiology) use endoscopes of different size and functionality. Therefore, the dimensions of embodiments of the present invention and the associated tasks may be modified to accommodate use of various endoscopes.

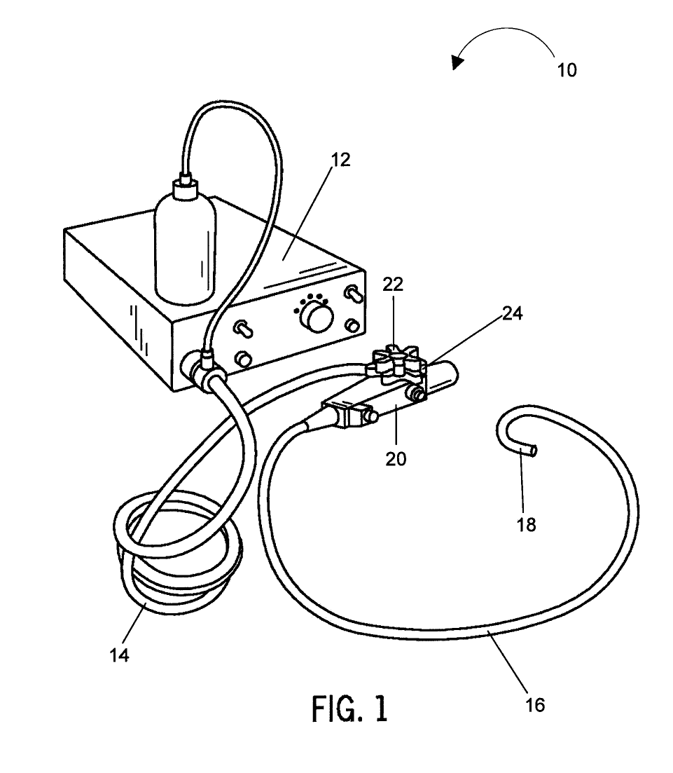

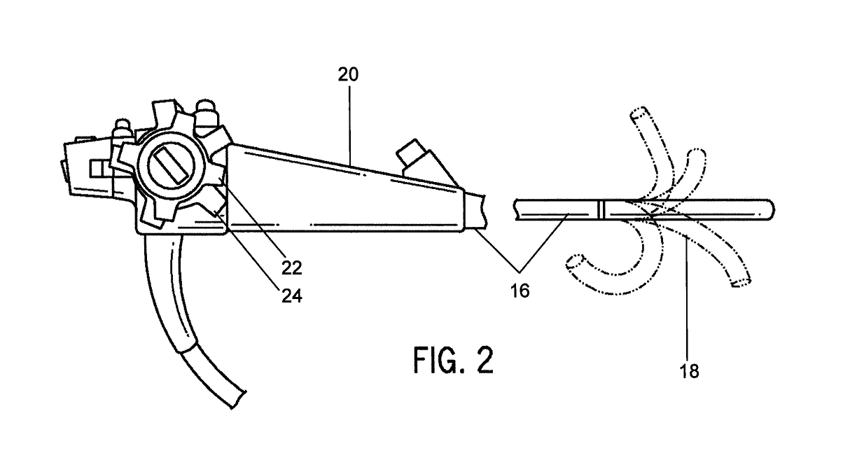

FIG. 1 illustrates an exemplary flexible endoscope system 10 that may be used with endoscopic training and assessment systems according to the invention. The flexible endoscope system 10 can include a control head 20 and a flexible endoscope insertion member 16 with a movable end 18. The control head 20 can be connected to a light source 12 via a connecting umbilical 14, through which pass other tubes (not shown) transmitting air, water, and suction. The control head 20 can include a small control knob 22 and large control knob 24 for maneuvering the movable end 18. The small and large control knobs 22, 24 can also be seen in FIG. 2, which illustrates an enlarged view of the control head 20 and movable end 18. The small control knob 22 can be manipulated in order to move the movable end 18 in a left or right direction, whereas the large control knob 24 moves the moveable end 18 in an upward or downward direction.

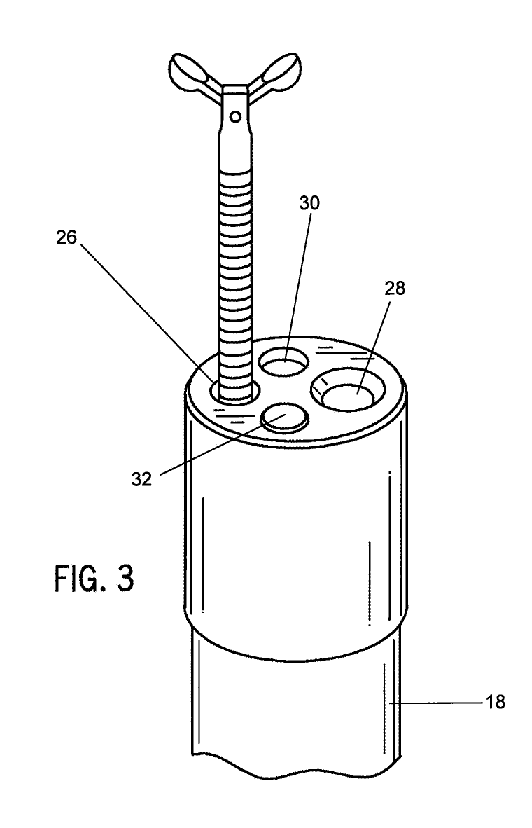

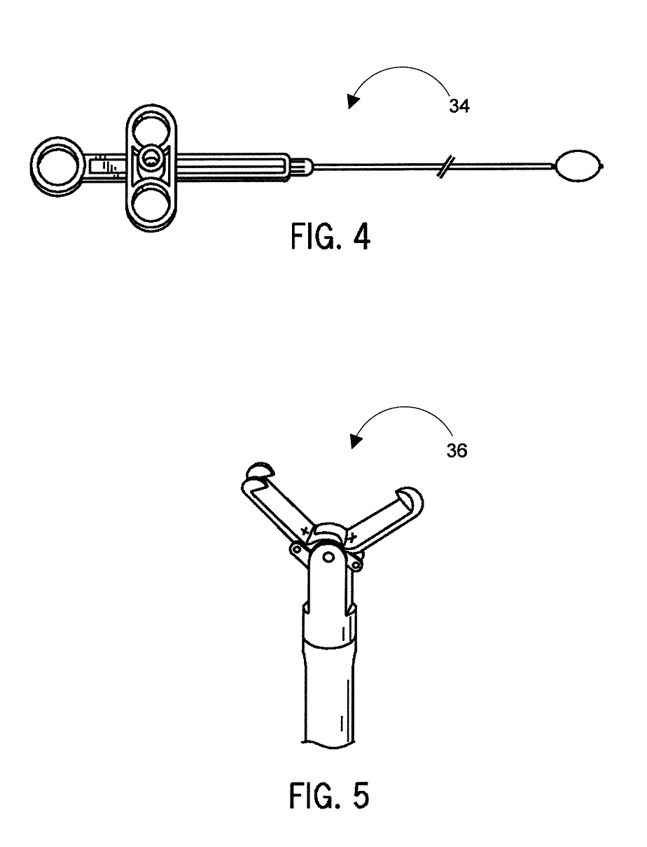

FIG. 3 illustrates the moveable end 18 of the flexible endoscope insertion member 16. The movable end 18 includes openings of a medical tool channel 26 and an air and water jet channel 28 in addition to a lens 30 and a light 32. Medical tools, such as a polypectomy snare 34 (FIG. 4) or a rat tooth forcep 36 (FIG. 5), can be inserted through the medical tool channel 26 for diagnostic and therapeutic procedures performed on a bodily feature. The medical tool can be seen in the field of view through the lens 30 and due to illumination provided by the light 32, while the air and water jet channel 28 are used for cleaning the lens 30. Further, the endoscope image is viewed on an eyepiece (not shown) or a display (not shown; for example, an LCD screen).

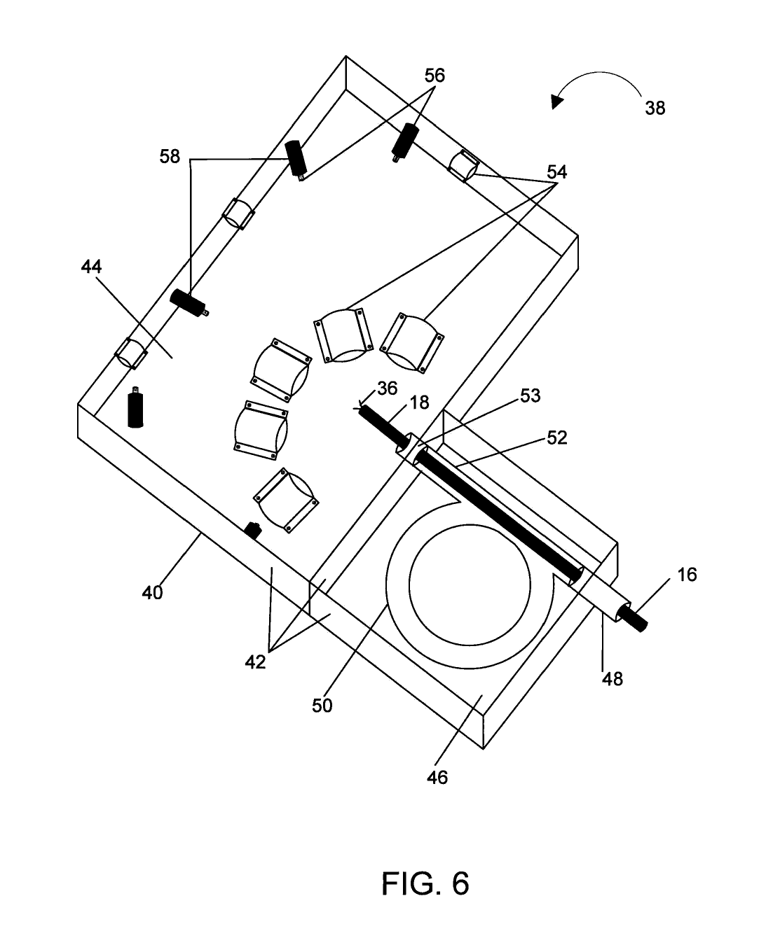

Turning now to FIG. 6, an endoscope training system 38 according to one embodiment of the invention facilitates training and assessment of a number of core cognitive and motor skills required for an endoscopist to be minimally competent in a routine colonoscopy. The core cognitive and motor skills for this task can include depth perception, navigation, and, loop reduction. Due to tight turns and redundancy in the colon areas, loops may form in the sigmoid or traverse colons that prevent the movable end 18 of the flexible endoscope insertion member 16 from advancing forward. As such the endoscopist may be required to perform loop reduction techniques.

The endoscopic training system 38 includes a portable part-task enclosure 40, made from a white Kydex material and having a plurality of walls 42. The walls 42 include a transparent cover disposed above a first internal chamber 46 and an opaque cover (omitted for illustrative purposes) disposed above a second internal chamber 44. The second internal chamber 44 can include a plurality of tubes 54 and a plurality of objects 58 hanging from support structures 56. The first internal chamber 46 can be connected to the second internal chamber 44 via a straight tube 53. The straight tube 53 is coupled to an open-topped straight channel or passage way 52 which is coupled to an open-topped elliptical shaped (as viewed from above) channel or passage way 50. Attached to one of the walls 42 is an access port 48 which couples the first internal chamber 46 to the external environment. The access port 48 can be configured to facilitate the passage of a flexible endoscope insertion member 16 through the elliptical shaped channel 50 and the straight channel 52.

Further, the straight channel 52 coupled to the elliptical shaped channel 50 of FIG. 6 advantageously has a cylindrical shape, with a diameter of 21 mm to best support the natural torque of the flexible endoscope insertion member 16. The straight channel 52 coupled to the elliptical shaped channel 50 can be made of a beige or sanshade high-density polyethylene (HDPE) material to allow the least resistance for the flexible endoscope insertion member 16 to pass through. The elliptical shaped channel 50 has a depth of 42 mm, to allow the flexible endoscope insertion member 16 to be flipped out from the elliptical shaped channel 50 during a loop reduction, but also to prevent the flexible endoscope insertion member 16 from flipping out prematurely while the loop is being formed.

Each of the plurality of tubes 54 of FIG. 6 can be formed by a soft, slippery material, such as insulation foam lined with satin-like fabric. This prevents the flexible endoscope insertion member 16 and movable end 18 from being damaged and also to pass easily there through. Further, the plurality of tubes 54 can be placed in an array in the second internal chamber 44 and as well as on the walls 42 of the second internal chamber 44, to form various color-coded pathways, so that various skills are required for navigation of the flexible endoscope insertion member 16 through the plurality of tubes 54.

Each of the plurality of objects 58 of FIG. 6 are made from a soft, flexible material and can have a single hole to allow hanging from the support structures 56. The support structures 56 may be, for example, elongated rods and made from a black, acrylonitrile butadiene styrene (ABS) material. The plurality of objects 58 advantageously have an inner diameter of 7.9 mm, a thickness of 2.6 mm and a length of 5.1 mm. As such, the objects are relatively small to allow retrieval by a grasper, such as a rat tooth forcep 36. The dimensions, materials, and placement of the straight channel 52, the elliptical shaped channel 50, the plurality of tubes 54, and the plurality of objects 58 also advantageously provide an appropriate training/assessment score discrepancy between novice and experienced endoscopists as described below.

In order to develop and assess navigation and loop reduction endoscopic skills, an endoscopist performs the following tasks using the flexible endoscope system 10 and the training system 38. The endoscopist begins by maneuvering the flexible endoscope insertion member 16 through the access port 48, into the straight channel 52, and further into the elliptical shaped channel 50, to form a loop within the first internal chamber 46. To further advance the flexible endoscope insertion member 16 into the second internal chamber 44, the endoscopist eliminates the loop of the flexible endoscope insertion member 16 by pulling and/or twisting the control head 20 about the axis of the insertion member 16. Once the flexible endoscope insertion member 16 is unlooped by flipping the flexible endoscope insertion member 16 out of the open-top surface of the elliptical shaped channel 50, the endoscopist can navigate the flexible endoscope insertion member 16 and movable end 18, by manipulating the small and large control knobs 22, 24, through one of the plurality of tubes 54. The endoscopist also manipulates the rat tooth forcep 36 to grasp one of the plurality of objects 58 at the end of the path. The endoscopist then removes the object 58 by maneuvering the flexible endoscope insertion member 16 back through the tube 54, through the straight tube 53, and through the straight channel 52 to the external environment. The above task is repeated until all objects 58 are removed from the portable part-task enclosure 40.

The endoscopist is given a predetermined time period to complete the above tasks. The total number of objects 58 removed from their respective support structure 56 and moved to the external environment during the time period are counted and converted to a quantitative score. The quantitative score can increase if all of the plurality of objects 58 are removed from the portable part-task enclosure 40 prior to expiration of the predetermined time period. For instance, the predetermined time period could be 5 minutes, each object 58 removed successfully could be worth 20 points, and each second remaining prior to the expiration of 5 minutes after all of the plurality of objects 58 are removed could be worth 1 point.

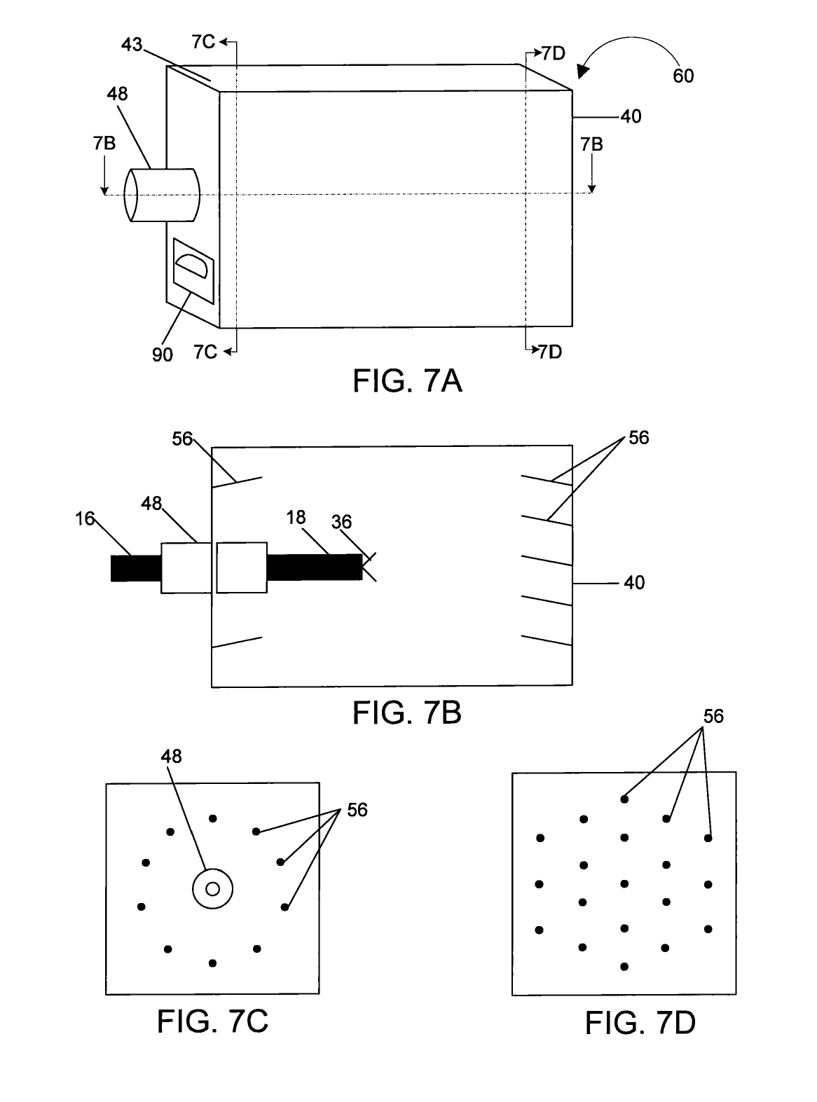

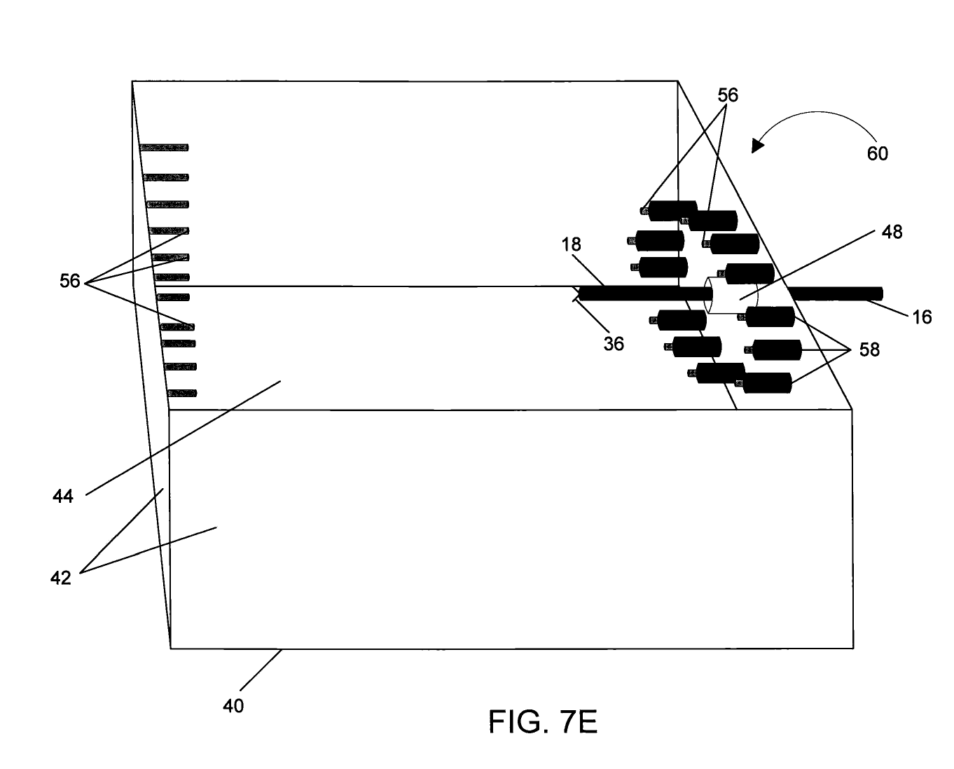

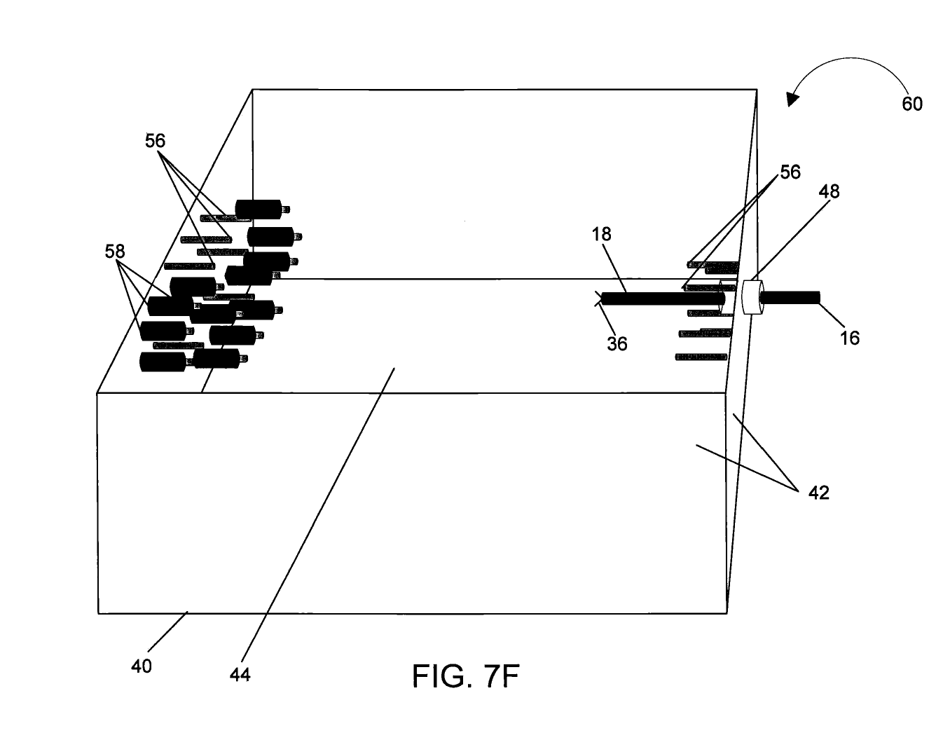

Turning now to FIGS. 7A-7F, an endoscopic training system 60 according to another embodiment of the invention requires an endoscopist to perform retroflexion, which is used in many procedures, including colonoscopy, sigmoidoscopy, and gastric cardiac biopsy. Retroflexion is considered a valuable part of the endoscopic examination of the large bowel, because it provides diagnostic yields of rectal lesions. Despite the benefits, retroflexion is still underperformed due to concerns about patient discomfort. Additionally, the technique may prove to be quite challenging for novices given that the orientation and the direction of motion is the reverse of what it is in a forward view. As a result, training fellows to perform retroflexion could lead to an improvement in both frequency and quality of such a maneuver being performed.

The endoscopic training system 60 for developing and assessing an endoscopist's retroflexion skills includes a portable part-task enclosure 40 and a plurality of walls 42 defining an internal chamber 44. Walls 42 include an opaque cover 43 (omitted in FIGS. 7E, 7F for illustrative purposes), which can be made from a white Kydex material, such that the internal chamber 44 is not visible through the walls 42. The internal chamber 44 can include a plurality of objects 58 hanging from support structures 56 on one of the plurality of walls 42. On an opposing wall 42 there can be a plurality of support structures 56 symmetrically arranged as seen in FIGS. 7C and 7D. Attached to one of the walls 42 is an access port 48 which couples the internal chamber 44 to the external environment. The access port 48, made from a black ABS material, can be configured to facilitate the passage of a flexible endoscope insertion member 16 to the internal chamber 44.

Further, the portable part-task enclosure 40 of FIGS. 7A-7F can have an advantageous length of 203.2 mm, and a width and height of 152.4 mm. This permits a flexible endoscope insertion member 16 to fully retroflex and straighten without being so spacious that the flexible endoscope insertion member 16 would fall under significant gravity, making the task unrealistic. The portable part-task enclosure 40 can be made from a white and black Kydex material.

Each of the plurality of support structures 56, as seen in FIG. 7B, can be slightly slanted upward at a 5 degree angle and made from a black ABS material. Also, each support structure 56 is 21 mm from the surface of the wall, and each of the plurality of objects 58 is 15 mm long, such that only one of the plurality of objects 58 can hang from each support structure 56. On the wall 42 containing the access port 48, the plurality of support structures, best seen in FIGS. 7E and 7F, are oriented at a distance so that full retroflexion of the flexible endoscope insertion member 16 is required to reach the plurality of objects 58. On the opposing wall 42, there can be more support structures 56 than the number of objects 58, allowing the endoscopist to choose from multiple support structures 56 on which to place one of the plurality of objects 58.

Each of the plurality of objects 58 of FIGS. 7E and 7F are made from a soft, flexible material and can have a single hole to facilitate hanging from the support structures 56. The plurality of objects 58 can have an inner diameter of 7.9 mm, a thickness of 2.6 mm, and a length of 15 mm, so they are relatively small to facilitate retrieval by a grasper, such as a rat tooth forcep 36. The dimensions, materials, and placement of portable part-task enclosure 40, the plurality of support structures 56, and the plurality of objects 58 also advantageously provide an appropriate training/assessment score discrepancy between novice and experienced endoscopists as described below.

In order to develop and assess retroflexion skills, an endoscopist performs the following tasks using the flexible endoscope system 10 and the training system 60. During the task shown in FIGS. 7E and 7F, the endoscopist inserts the flexible endoscope insertion member 16 through access port 48 and maneuvers the movable end 18 by fully turning the large control knob 24 counterclockwise. This movement causes the movable end 18 to bend backwards, resulting in retroflexion. The endoscopist can use the rat tooth forcep 36 that is extended through the flexible endoscope insertion member 16 to grasp one of the plurality of objects 58 and remove it from the support structure 56 coupled to the wall 42 containing the access port 48 (see FIG. 7E). The endoscopist can then straighten the flexible endoscope insertion member 16 and movable end 18, again by controlling the small and large control knobs 22, 24, while still grasping the object 58, and then place it on a support structure on the opposing wall 42 (see FIG. 7F). The above task is repeated until all objects 58 are moved from the support structures on one wall 42 to the support structures on an opposing wall 42.

The endoscopist is given a predetermined time period to complete the above task. The total number of objects 58 transferred from the wall 42 containing the access port 48 to the opposing wall 42 are counted and converted to a quantitative score. The quantitative score can increase if all of the plurality of objects 58 is transferred from the wall 42 containing the access port 48 to the opposing wall 42 prior to expiration of the predetermined time period. If any of the plurality of objects 58 is dropped within the internal chamber 44, they can be ignored and retrieved if the time period has not expired before moving the other objects 58. For instance, the predetermined time period could be 5 minutes, each object 58 transferred successfully could be worth 10 points, and each second remaining prior to the expiration of 5 minutes after all of the plurality of objects 58 are transferred could be worth 1 point.

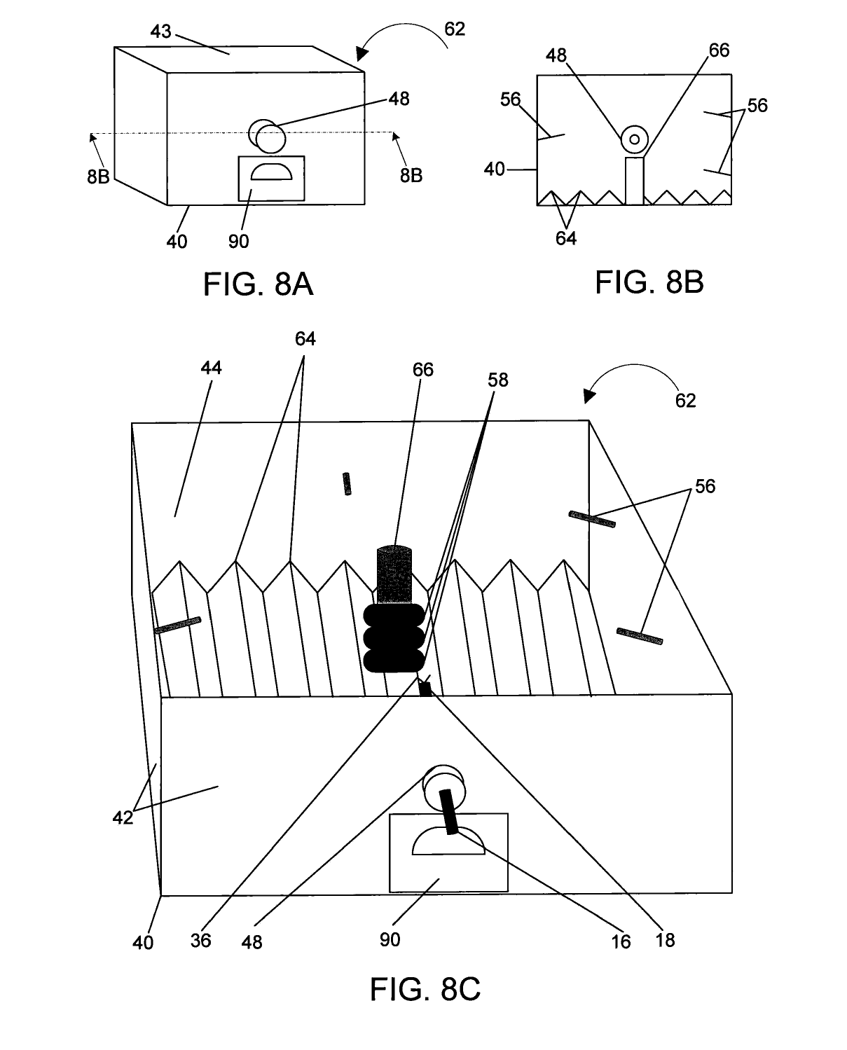

Turning now to FIGS. 8A-8C, an endoscopic training system 62 according to another embodiment of the invention facilitates training and assessment of a variety of skill sets including torquing of the flexible endoscope insertion member, precise targeting, and depth perception. These skills are used in many endoscopic procedures, including colonoscopy. While the tasks described in previous embodiments focus on an individual skill set, such as loop reduction and retroflexion, this embodiment aims to simulate the combined use of these skills in a clinical setting.

The endoscopic training system 62 for developing and assessing an endoscopist's torquing, precise targeting, and depth perception skills includes a portable part-task enclosure 40 and a plurality of walls 42 defining an internal chamber 44. Walls 42 include an opaque cover 43 (omitted in FIG. 8C for illustrative purposes), which can be made from a white, Kydex material, such that the internal chamber 44 is not visible through the walls 42. The internal chamber 44 can include a plurality of objects 58 stacked on a central post 66 which can be centrally coupled to an undulated floor 64. On the plurality of walls 42 there can be a plurality of support structures 56, which can be made from a black ABS material and arranged as seen in FIGS. 8B and 8C. Attached to one of the walls 42 is an access port 48 which can be made from a black ABS material and couples the internal chamber 44 to the external environment. The access port 48 can be configured to facilitate the passage of a flexible endoscope insertion member 16 to the internal chamber 44.

Further, the portable part-task enclosure 40 of FIGS. 8A-8C can have an advantageous length of 203.2 mm, and a width and height of 152.4 mm. The distance between the central post 66 and the access port 48 is 76.2 mm, and the distance between the central post 66 and the side walls 42 is 101.6 mm, positioning the central post 66 directly in the middle of the undulated floor 64. These distances allow the flexible endoscope insertion member 16 to have enough space within the portable part-task enclosure 40 to manipulate the plurality of objects 58. However, the chamber is not so spacious that the flexible endoscope insertion member 16 would fall under significant gravity, making the task unrealistic. The portable part-task enclosure 40 can be made from a black and white Kydex material.

The undulated floor 64 (that is, a floor having an array of side-by-side v-shaped surfaces as viewed from the side) shown in FIGS. 8B and 8C, reduces the difficulty of grasping and lifting accidentally dropped objects 58. The undulated floor 64 can be made from a beige HDPE material. The amplitude of the undulated floor is 10.53 mm, and it can cover the entire bottom wall 42 of the portable part-task enclosure 40.

The central post 66, seen in FIGS. 8B and 8C, is 60.03 mm tall from the floor to advantageously allow all of the plurality of objects 58 to be stacked on the central post 66 at once without obstructing the endoscopist's view. The central post 66 is 14.28 mm in diameter and can be made from a black ABS material. Further, each of the plurality of support structures 56, as seen in FIGS. 8B and 8C, can be slightly slanted upward at a 25 degree angle and coupled to the plurality of walls 42. Each of the plurality of objects 58 in FIG. 8C can have a single hole to allow hanging from the support structures 56.

Each of the plurality of objects 58, shown in FIG. 8C, can be ring shaped and can have an inner diameter of 0.94 in, an outer diameter of 1.16 in, and a thickness of 0.13 in, so they are relatively small to facilitate retrieval by a grasper, such as a rat tooth forcep 36. As such, if one of the plurality of objects 58 is dropped, it always has a portion that protrudes above the undulated floor 64. This allows an endoscopist to easily re-grasp the object 58 on the undulated floor 64 with the rat tooth forcep 36. The ring shaped objects 58 can be made from a silicone material, which facilitates better grasping by forceps due to its compliance. The dimensions, materials, and placement of the portable part-task enclosure 40, the central post 66, the plurality of support structures 56, and the plurality of objects 58 also advantageously provide an appropriate training/assessment score discrepancy between novice and experienced endoscopists as described below.

In order to develop and assess torquing, precise targeting, and depth perception skills, an endoscopist performs the following tasks using the flexible endoscope system 10 and the training system 62. During the task, best shown in FIG. 8C, the endoscopist inserts the flexible endoscope insertion member 16 through the access port 48 and maneuvers the movable end 18 by controlling the small and large control knobs 22, 24 of the control head 20 as seen in FIGS. 1 and 2, and torquing the flexible endoscope insertion member 16. The endoscopist can use the rat tooth forcep 36 to grasp one of the plurality of objects 58, remove it from the central post 66, and place it on one of the support structures 56. The endoscopist repeats this procedure, transferring the plurality of objects 58, each to a different support structure, until no objects 58 remain on the central post 66. Lastly, the endoscopist transfers the plurality of objects 58, one at a time, from the support structures 56 to the central post 66 in the exact same order the objects 58 were initially transferred from the central post 66 to the support structures 56. Any accidentally dropped objects 58 are picked up with the rat tooth forcep 36 and transferred to its target support structure 56 or central post 66 before proceeding to the next ring shaped object 58.

The endoscopist is given a predetermined time period to complete the above task. The total number of objects 58 transferred from the central post 66 to a support structure 56 and from a support structure 56 back to the central post 66 are counted and converted to quantitative score. The quantitative score can increase if all of the plurality of objects 58 are transferred from the central post 66 to the support structures 56 and back to the central post 66 prior to expiration of the predetermined time period. For instance, the predetermined time period could be 5 minutes, each object 58 transferred successfully could be worth 10 points, and each second remaining prior to the expiration of 5 minutes after all of the plurality of objects 58 are transferred back to the central post 66 could be worth 1 point.

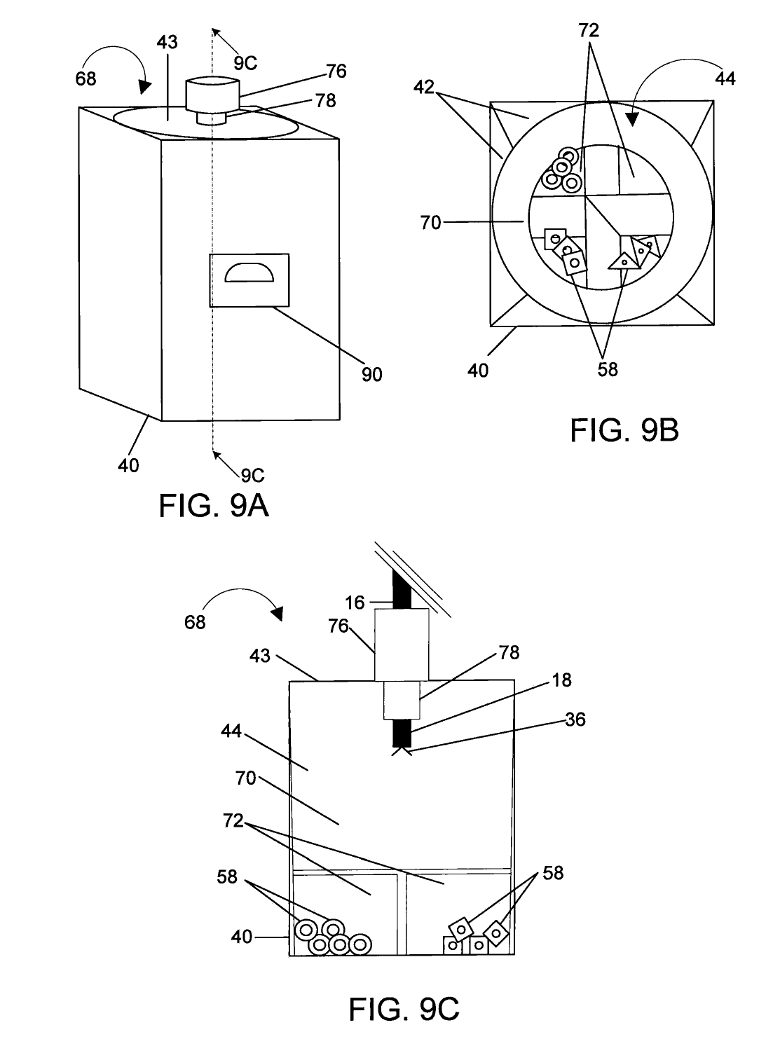

Turning now to FIGS. 9A-9C, an endoscopic training system 68 according to another embodiment of the invention requires an endoscopist to make fine adjustments to the moveable end 18 of the flexible endoscope insertion member 16 by manipulating the small and large control knobs 22, 24, both separately and in combination. This precision is valuable in almost all diagnostic and therapeutic endoscopic examinations, including colonoscopies. The task described below in relation to tip deflection facilitates training these skills in order to improve precision during the endoscopic procedures.

The endoscopic training system 68 for developing and assessing an endoscopist's tip deflection skills includes a rectangular-shaped portable part-task enclosure 40 and a plurality of walls 42 defining an internal chamber 44. Walls 42 include an opaque cover 43 (omitted in FIG. 9B for illustrative purposes), which can be made from a white Kydex and ABS material or PVC, such that the internal chamber 44 is not visible through the walls 42. The internal chamber 44 can include a bin 70 coupled to the bottom wall of the plurality of walls 42. The bin 70 can be divided into four equal-sized compartments 72 which can hold a plurality of objects 58 (see FIGS. 9B and 9C). Attached to the opaque cover 43 is a long access port 78 which couples the internal chamber 44 to the external environment. The long access port 78 can be configured to facilitate the passage of a flexible endoscope insertion member 16 to the internal chamber 44. The long access port 78 can also have a lock mechanism 76 coupled to it so only the moveable end 18 of the flexible endoscope insertion member 16 can move freely.

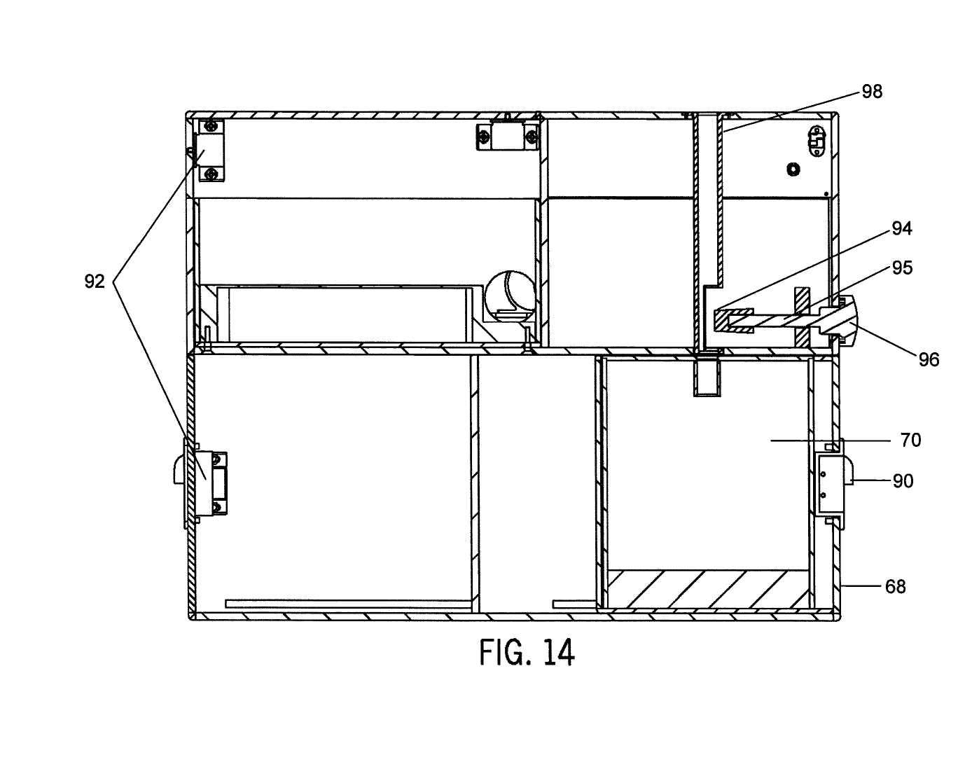

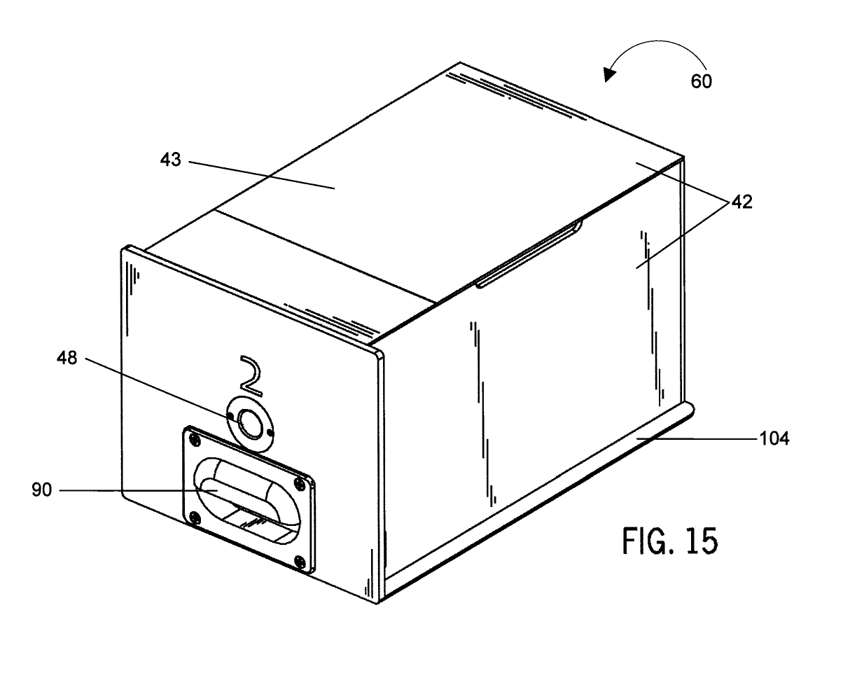

A side perspective view of the lock mechanism 76 can be seen in FIG. 14. The lock mechanism includes a silicon end cap 94 coupled to an adjustment knob 96 and a guide tube 98. The lock mechanism is disposed on a top portion of the portable part-task enclosure 40. The adjustment knob 96 is coupled to a silicon end cap 94 by a screw-like threaded stem 95. As such, when the adjustment knob 96 is turned in a clockwise direction, the silicon end cap 94 presses against the flexible endoscope insertion member 16, which is inserted through the guide tube 98 and long access port 78, thereby securing it in place.

Further, the portable part-task enclosure 40 of FIGS. 9A-9C advantageously has a length of 153.2 mm, a width of 140.75 mm, and a height of 164.3 mm. The long access port 78 has a length of 158.1 mm. The dimensions of the portable part-task enclosure 40 and the long access port 78 permit the moveable end 18 of the flexible endoscope insertion member 16 to move freely, while the rest of the flexible endoscope insertion member 16 is fixed by the compression lock mechanism 76. This inhibits the insertion member 16 from being moved and damaged throughout the task. The portable part-task enclosure 40 can be made from a black and white Kydex material.

The bin 70, seen in FIGS. 9B and 9C, advantageously has a diameter of 133.35 mm and height of 165.58 mm, and it can be made from a white acrylic material. The bin 70 is divided equally into quadrants as seen in FIG. 9B and each quadrant can contain a plurality of objects 58.

Each of the plurality of objects 58, seen in FIGS. 9B and 9C, can be of various geometric shapes, but all having a hole with rims in the middle and inner diameter of 4.7 mm. The geometric shapes of the plurality of objects 58 can include a barrel shape, a rectangular block, or a triangular shape. The barrel shaped objects 58 can have an outer diameter of 10.3 mm at its thickest and 6.6 mm at its thinnest. The rectangular block objects 58 can have a length and width of 14.4 mm, and a height of 19.7 mm. The triangular shaped objects 58 can have sides of equal length of 18.5 mm and a height of 14.3 mm. All of the plurality of objects are relatively small to allow optimal retrieval by a grasper, such as a rat tooth forcep 36, and can be made from a Delrin material. The dimensions, materials, and placement of portable part-task enclosure 40, the bin 70 and its compartments 72, and the plurality of objects 58 also advantageously provide an appropriate training/assessment score discrepancy between novice and experienced endoscopists as described below.

In order to develop and assess tip deflection skills, an endoscopist performs the following tasks using the flexible endoscope system 10 and the training system 68. Prior to the predetermined time period starting, each type of geometric shaped objects 58 are placed into their own compartment 72 of the bin 70, leaving one compartment 72 empty as seen in FIG. 9B. During the task, the endoscopist inserts the flexible endoscope insertion member 16 through the long access port 78 and maneuvers the movable end 18 by only controlling the small and large control knobs 22, 24 of the control head 20. The shaft of the endoscope may not be touched. The endoscopist advances and retracts the rat tooth forcep 36 and opens and closes it in order to grasp and transfer an object 58 from its original compartment 72 to an empty compartment 72. All of the objects 58 of one geometric shape are transferred from their compartment 72 to an empty compartment 72 before the endoscopist can begin transferring the next type of geometric shaped objects 58 to the newly empty compartment 72. The endoscopist repeats this procedure until all the different types of geometric shaped objects 58 are moved from their original compartment 72 to an empty compartment 72. Any accidentally dropped objects 58 are picked up with the rat tooth forcep 36 and transferred to its target compartment 72 before proceeding to the next object 58.

The endoscopist is given a predetermined time period to complete the above task. The total number of objects 58 transferred from their initial compartment 72 to an empty compartment 72 are counted and converted to a quantitative score. The quantitative score can increase if all of the plurality of objects 58 are transferred from their initial compartment 72 to an empty compartment 72, in the manner described above, prior to expiration of the predetermined time period. For instance, the predetermined time period could be 5 minutes, each object 58 transferred successfully could be worth 10 points, and each second remaining prior to the expiration of 5 minutes after all of the plurality of objects 58 are transferred from their initial compartment 72 to an empty compartment 72 could be worth 1 point.

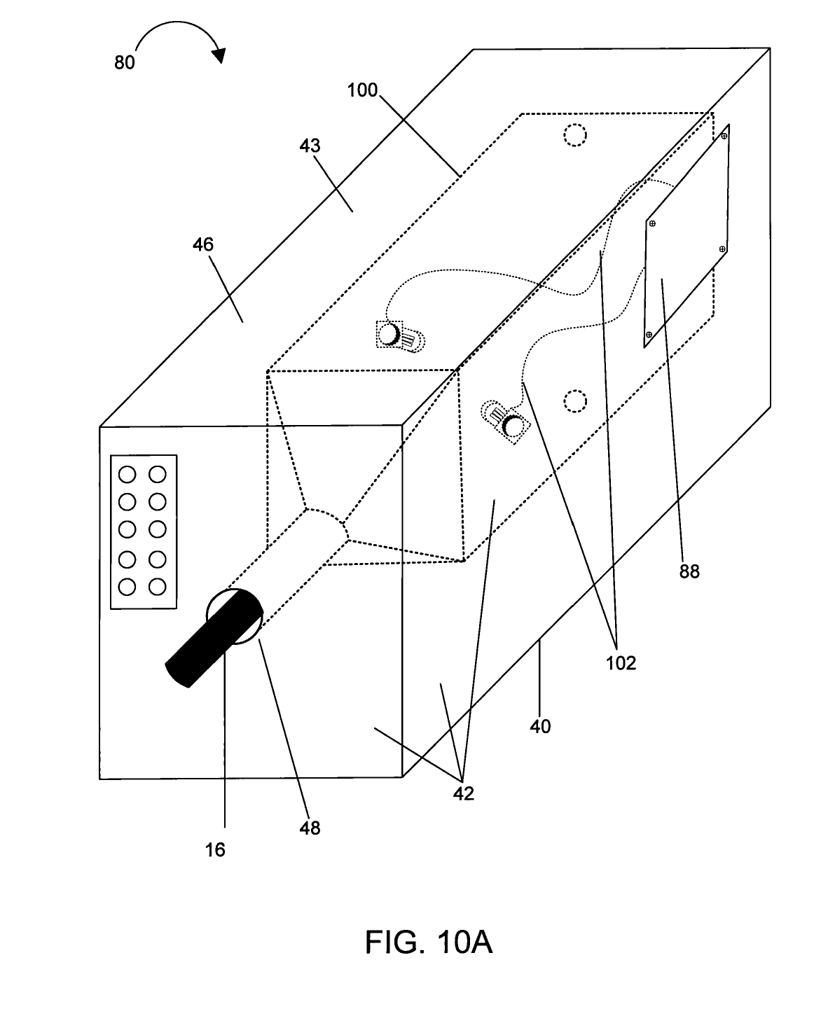

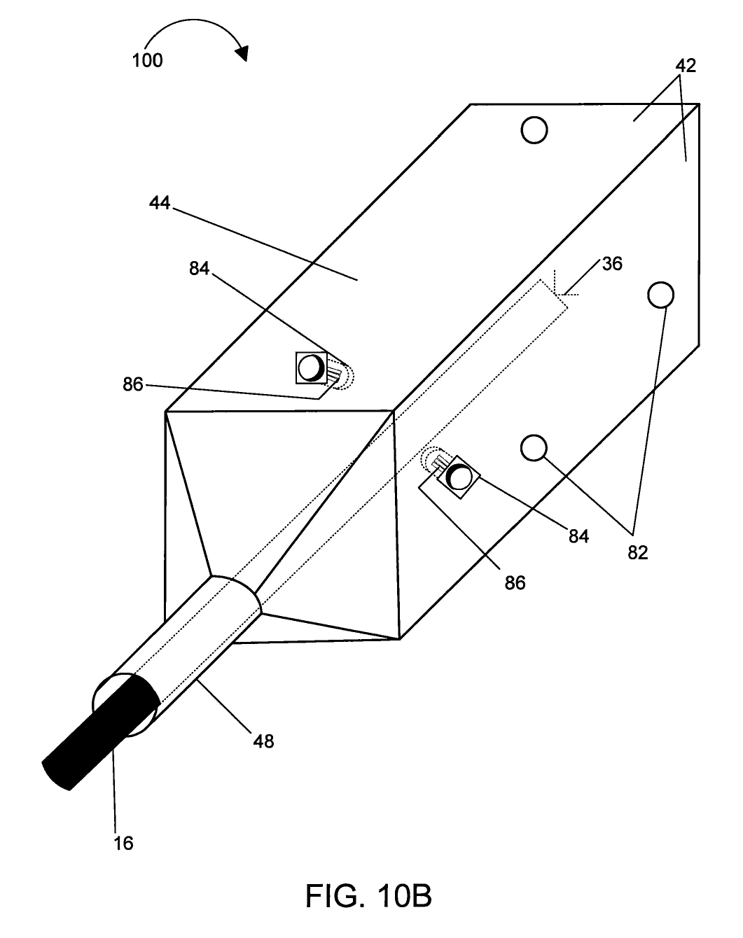

Turning now to FIGS. 10A and 10B, an endoscopic training system 80 according to another embodiment of the invention requires an endoscopist to perform a simulated snare polypectomy, which is preferred when a polyp is 1 cm or greater in size, and can be done during a colonoscopy. The ability to perform polypectomy is valuable for all endoscopists because they reduce the risk of colon cancer.

The endoscopic training system 80 for developing and assessing an endoscopist's polypectomy skills includes a rectangular shaped portable part-task enclosure 40 and a plurality of walls 42 defining a first internal chamber 46, and a second enclosure 100 disposed in the portable part-task enclosure 40. The walls 42 defining the second internal chamber 44 can include holes 82 through which indicators 84 extend into the second internal chamber 44, and each indicator 84 is coupled to a load sensor 86 (see FIG. 10B). A controller 88 can operatively connect the indicators 84 and load sensors 86 with connectors 102. Attached to a side wall of the plurality of walls 42 is an access port 48 which couples the internal chamber 44 to the external environment. The access port 48, which can be made from a black ABS material, can be configured to facilitate the passage of a flexible endoscope insertion member 16 to the internal chamber 44.

Further, the portable part-task enclosure 40 of FIGS. 10A and 10B has a length of 299 mm, a width of 156.48 mm, and a height of 145.81 mm. The second enclosure 100 advantageously has a length of 231.8 mm, a width and height of 81.15 mm. The second enclosure includes a tapered end that leads to the access port. The dimensions of the second enclosure 100 provide varying difficulty depending on the angle of the surface on which the indicator 84 is disposed. Further, the dimensions allow enough space for the flexible endoscope insertion member 16 to be manipulated in different directions, including full retroflexion for the indicators 84 located on the wall 42 proximate the access port 48. The portable part-task enclosure 40 can be made from a white 3D printed material.

Each indicator 84 simulates a polyp and can be an LED light encased in a silicone shell that extends through one of the holes 82 on the plurality of walls 42 of the second enclosure 100. The LED indicators 84 can be shaped to simulate clinical polyps and also so the endoscopist is forced to snare the LED indicators 84 at the base with the polypectomy snare 34, rather than grabbing it elsewhere. The LED indicators 84 are dispersed throughout the plurality of walls 42 coupled to the holes 82 in a random fashion.

The controller 88 can be a microcontroller controlling the LED indicators 84 so that when snared by the polypectomy snare 34 with a predetermined amount of force sensed by the load sensor 86, the LED indicators 84 would flash, and after a certain about of time, the LED indicators 84 could stay illuminated. The dimensions, materials, and placement of the portable part-task enclosure 40, the second enclosure 100, the indicators 84, and the amount of time an LED indicator 84 flashes after being snared also advantageously provide an appropriate training/assessment score discrepancy between novice and experienced endoscopists as described below.

In order to develop and assess polypectomy skills, an endoscopist performs the following tasks using the flexible endoscope system 10 and the training system 80. Prior to the tasks, the controller 88 is reset so that all the LED indicators 84 are not illuminated. During the task, the endoscopist inserts the flexible endoscope insertion member 16 through the access port 48 and maneuvers the movable end 18 by controlling the small and large control knobs 22, 24 of the control head 20. The endoscopist uses a polypectomy snare 34 inserted through the medical tool channel 26 of the flexible endoscope insertion member 16 and moveable end 18 in order to capture an LED indicator 84 at the base. The endoscopist can snare the LED indicator 84 by opening the polypectomy snare 34 over the LED indicator 84, then closing the polypectomy snare 34 around the base to simulate resection. Once the LED indicator 84 is properly snared and a sufficient force is applied, it will flash and stay illuminated. The LED indicators 84 can be snared in any order, one at a time.

The endoscopist is given a predetermined time period to complete the above task. The total number of LED indicators 84 that are successfully snared or illuminated are counted and converted to a quantitative score. The quantitative score can increase if all of the LED indicators 84 are snared prior to expiration of the predetermined time period. For instance, the predetermined time period could be 5 minutes, each LED indicator 84 snared successfully could be worth 10 points, and each second remaining prior to the expiration of 5 minutes after all of the LED indicators 84 are snared could be worth 1 point.

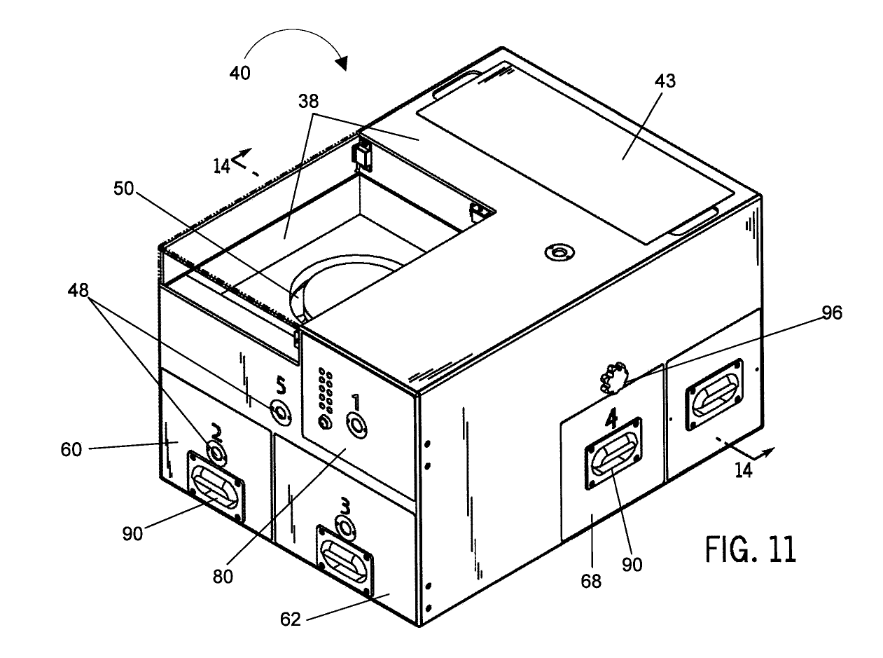

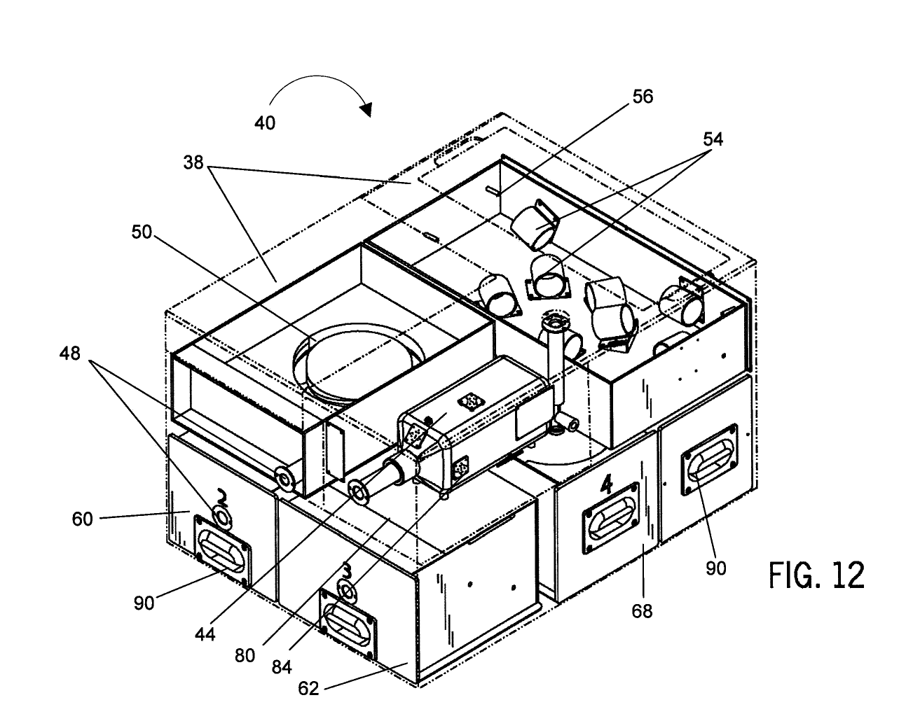

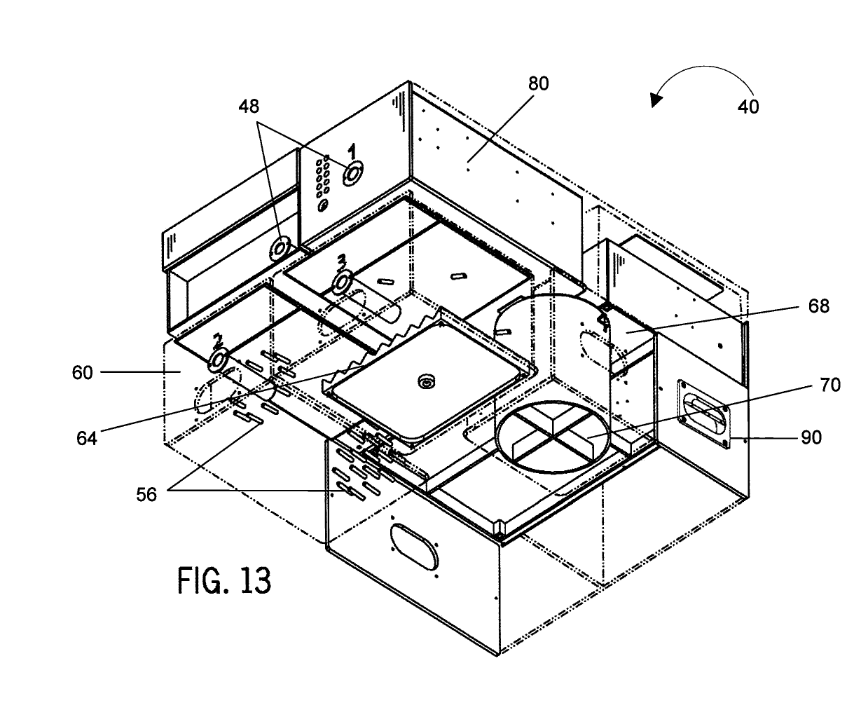

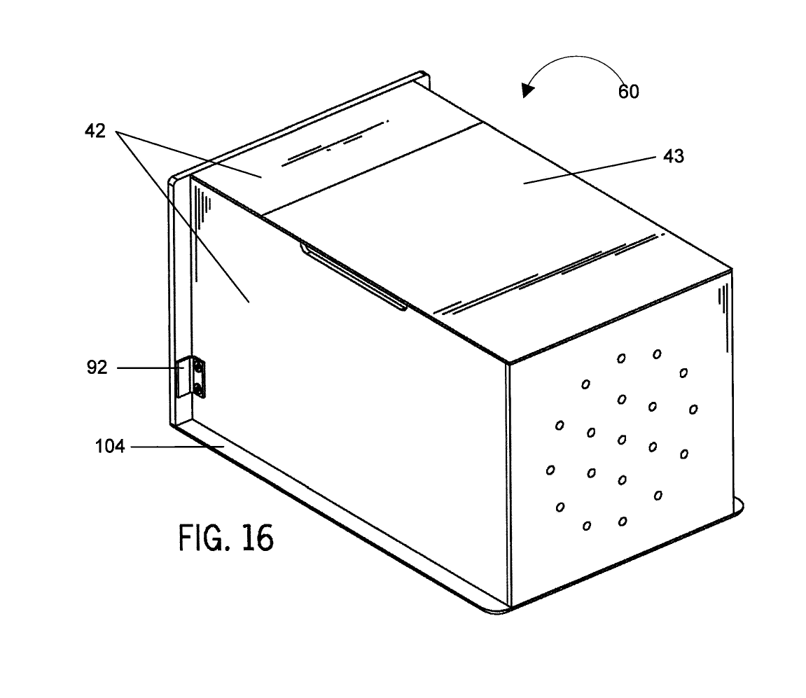

Turning to FIG. 11, another embodiment of the present invention could include each of the endoscopic training systems 38, 60, 62, 68, and 80 arranged in a single portable part-task enclosure or “kit” 40. Looking at FIGS. 12 and 13, some of the walls 42 are transparent to show a possible arrangement of all the endoscopic training systems 38, 60, 62, 68, and 80 in the single portable part-task enclosure 40. FIG. 12 shows the opaque cover 43 of the endoscopic training system 38, which can be removed to reset the plurality of objects 58. Other endoscopic training systems, such as 60, 62, 68, and 80, have similar removable opaque covers 43 for resetting the plurality of objects 58. Some of the endoscopic training systems, specifically 60, 62, and 68, are drawer-like structures, shown in FIGS. 15 and 16, to facilitate removing the covers 43 and resetting objects 58. The drawer-like structures include a handle 90 for removing the endoscopic training system from the portable part-task enclosure 40, and guides 104 for assisting the user when inserting or removing the drawer-like structure from the portable part-task enclosure 40. The drawer-like structures can also include guards 92 for protecting one of the walls 42 of the endoscopic training system when inserting into the portable part-task enclosure 40. The guards 92 also help prevent the drawer-like structure from being pushed too far into the portable part-task enclosure 40.

Other embodiments of the present invention could also include an endoscopic training system where the task includes dot connecting by coupling a pencil lead to the movable end 18 of the flexible endoscope insertion member 16. The endoscopist could be required to use the small or large control knobs 22, 24 to maneuver the movable end 18 of the flexible endoscope insertion member 16 in order to draw a continual line to connect dots of various configurations. Another version of this embodiment could include a touch screen with a stylus.

Another embodiment of the present invention could also include an endoscopic training system where the endoscopist is required to navigate the movable end 18 of the flexible endoscope insertion member 16 in three-dimensional space through a maze. This could be used to train endoscopists how to navigate by visualization. Other iterations could involve using the moveable end 18 of the flexible endoscope insertion member 16 to push a ball or object through the maze.

Yet another embodiment of the present invention could include an endoscopic training system where the endoscopist is required to navigate the movable end 18 of the flexible endoscope insertion member 16 through ring shaped objects 58 attached to different walls 42 that define an internal chamber 44. The endoscopist could also be required to collect the ring shaped objects 58 around the flexible endoscope insertion member 16.

Lastly, in another embodiment of the present invention could also include an endoscopic training system which includes cylinder shaped objects 58 that have a post sticking out from one end and a hole receptacle on the other end. The endoscopist could be required to retroflex the flexible endoscope insertion member 16 to grab each cylinder shaped object 58, rotate the flexible endoscope insertion member 16, and then stack each of the cylinder shaped objects 58 on one another.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

1. An endoscopic training system for development of endoscopic procedure skills and assessment of technical competency of an endoscopist, the endoscopic training system comprising:
a portable part-task enclosure including a plurality of walls defining an internal chamber, at least one of the plurality of walls including an access port coupling the internal chamber to an external environment, the access port being configured to facilitate passage of a flexible endoscope insertion member;
a plurality of objects disposed within the internal chamber, the plurality of objects each being configured for manipulation by a medical tool extending through the flexible endoscope insertion member.

2. The endoscopic training system as recited in claim 1 further comprising a plurality of support structures fixed to at least one of the plurality of walls of the portable part-task enclosure and movably supporting the plurality of objects disposed within the internal chamber.

3. The endoscopic training system as recited in claim 2 wherein the plurality of support structures are poles.

4. The endoscopic training system as recited in claim 1 wherein the plurality of objects are indicators providing an indication upon manipulation by the medical tool.

5. The endoscopic training system as recited in claim 4 wherein a load sensor is coupled to each indicator.

6. The endoscopic training system as recited in claim 5 wherein a controller operatively connects to the indicators and the load sensors to illuminate one of the indicators when a predetermined load is applied to one of the indicators by the medical tool and sensed by one of the load sensors.

7. The endoscopic training system as recited in claim 1 further comprising a bin coupled to a bottom wall of the plurality of walls; the bin being configured to receive the plurality of objects.

8. The endoscopic training system as recited in claim 7 wherein the bin is divided into a plurality of compartments.

9. The endoscopic training system as recited in claim 1 further comprising a compression lock mechanism coupled to a top wall of the plurality of walls, the compression lock mechanism allowing only a moveable end of the flexible endoscope insertion member to move freely within the internal chamber.

10. The endoscopic training system as recited in claim 1 wherein at least one of the plurality of walls is opaque.

11. A method for development of endoscopic procedure skills and assessment of technical competency of an endoscopist, the steps of the method comprising:
providing a portable part-task enclosure having a plurality of walls defining an internal chamber, the internal chamber having a plurality of objects disposed therein, and the portable part-task enclosure having an access port coupling the internal chamber to an external environment;
inserting a flexible endoscope insertion member through the access port and into the internal chamber;
maneuvering the flexible endoscope insertion member within the internal chamber;
delivering a medical tool to the internal chamber via the flexible endoscope insertion member; and
manipulating at least one of the plurality of objects within the internal chamber using the medical tool.

12. The method as recited in claim 11 wherein the portable part-task enclosure further includes an elliptical shaped channel, and further comprising the steps of:
looping the flexible endoscope insertion member through the elliptical shaped channel, to form a loop;
performing a loop reduction technique by simultaneously pulling and twisting the flexible endoscope insertion member to eliminate the loop; and
navigating the flexible endoscope insertion member to manipulate at least one of the plurality of objects with the medical tool.

13. The method as recited in claim 11 further comprising a plurality of support structures fixed to at least one of the plurality of walls of the portable part-task enclosure, the plurality of support structures movably supporting the plurality of objects, and further comprising the steps of:
bending a movable end of the flexible endoscope insertion member backwards to retroflex the flexible endoscope insertion member within the internal chamber; and
while retroflexed, manipulating at least one of the plurality of objects with the medical tool.

14. The method as recited in claim 13 further comprising the step of moving at least one of the plurality of objects with the medical tool from at least one of the support structures to at least one different support structure.

15. The method as recited in claim 11 wherein the plurality of objects include a plurality of support structures fixed to at least one of the plurality of walls of the portable part-task enclosure, a post coupled to a bottom wall of the plurality of walls, and further comprising the steps of:
twisting the flexible endoscope insertion member to torque the flexible endoscope insertion member within the internal chamber; and
while torqued, manipulating at least one of the plurality of objects with the medical tool.

16. The method as recited in claim 15 further comprising the step of moving at least one of the plurality of objects with the medical tool from the post to at least one of the support structures.

17. The method as recited in claim 11 wherein the portable part-task enclosure includes a bin divided into a plurality of compartments, the bin coupled a bottom wall of the plurality of walls, and further comprising the steps of:
controlling only a moveable end of the flexible endoscope insertion member to deflect the moveable end within the internal chamber; and
while deflected, manipulating at least one of the plurality of objects with the medical tool.

18. The method as recited in claim 17 further comprising the step of moving at least one of the plurality of objects with the medical tool from at least one of the compartments to at least one different compartment.

19. The method as recited in claim 11 wherein the plurality of objects are indicators disposed on the plurality of walls, and further comprising the steps of:
navigating a medical tool, the medical tool being a polypectomy snare, to capture at least one indicator around its base; and
snaring the indicator at the base with the polypectomy snare, to illuminate the indicator.

20. The method as recited in claim 11 further comprising the step of quantitatively scoring an endoscopist based on a number of the plurality of objects manipulated within the portable part-task enclosure during a predetermined time period and increasing the quantitative score when all of the plurality of objects are manipulated prior to expiration of the predetermined time period.

21. The method as recited in claim 11 wherein the medical tool is a rat tooth forcep.

22. An endoscopic training kit for development of endoscopic procedure skills and assessment of technical competency of an endoscopist, the endoscopic training kit comprising:
a first portable part-task enclosure including a first plurality of walls defining a first internal chamber, at least one of the plurality of walls including a first access port coupling the internal chamber to an external environment, the first access port being configured to facilitate passage of a flexible endoscope insertion member;
a plurality of objects disposed within the first internal chamber, the plurality of objects each being configured for manipulation by a medical tool extending through the flexible endoscope insertion member;
a second portable part-task enclosure including a second plurality of walls defining a second internal chamber, at least one of the second plurality of walls including a second access port coupling the second internal chamber to the external environment, the second access port being configured to facilitate passage of the flexible endoscope insertion member; and
an object disposed within the second internal chamber, the object defining a passageway for maneuvering therethrough by the flexible endoscope insertion member.
